(19) United States
(12) Patent Application Publication
(10) Pub. No.: US 2015/0127073 A1
(43) Pub. Date: May 7, 2015

(54) DEVICE FOR EXTERNAL MEDICAL TREATMENT USING LIGHT OF VARYING PULSE LENGTHS

(71) Applicant: BIOLIGHT PATENT HOLDING AB, Stockholm (SE)

(72) Inventor: Rolf Thiberg, Akersberga (SE)

(73) Assignee: BIOLIGHT PATENT HOLDING AB, Stockholm (SE)

(21) Appl. No.: 14/397,597

(22) PCT Filed: Apr. 26, 2013
(86) PCT No.: PCT/SE2013/050467

§ 371 Date: Oct. 28, 2014

(30) Foreign Application Priority Data
May 7, 2012 (SE) 1250461-9

(51) Int. Cl. A61N 5/06

(52) U.S. Cl. CPC A61N 5/06; A61N 2005/0643; A61N 2005/0626; A61N 2005/0651; A61N 2005/0659; A61N 2005/0662; A61N 2005/0644
USPC 607/90

(57) ABSTRACT

Device for external medical treatment using light, including a light emitting element arranged to abut against or near the body of an individual and a drive device for the light emitting element, which includes light emitting diodes. The drive device controls the light emitting element to emit light of one or several wavelengths during one or several predetermined times, and to emit the light in a pulsating manner. The light emitting element includes a cover and a light emitting diode plate, supporting the light emitting diodes positioned thereon with a certain distance between two adjacent light emitting diodes. The drive device is arranged to control the light emitting element to emit pulsating light with a pulse length which lies within an interval of about 60% to 90% of the time between the respective start edge of two consecutive pulses, and to vary the length of the pulses over time.

The present invention relates to a device for external medical treatment using light.

In for instance the Swedish patent no 502 784, a device for external medical treatment using light is disclosed, comprising a light emitting means arranged to abut against or be held near the body of an individual, as well as a drive device for the light emitting means, which light emitting means comprises light emitting diodes or the corresponding, and is arranged to emit infrared light. The invention according to the said patent specifies that the drive device is arranged to control the light emitting means to, in a first step, emit infrared light during a first predetermined time, and to thereafter, in a second step, emit visible light during a second predetermined time, and that the drive device is arranged to control the light emitting means to emit the infrared light and the visible light, respectively, in a pulsating manner according to a predetermined series of pulse frequencies.

It is also known to emit also other monochromatic lights for treatment of various medical conditions.

Furthermore, it has turned out that treatment with only one or several monochromatic lights and other than infrared lights, such as visible light of various colours, which is emitted according to a certain pulse frequency, results in very good treatment results.

It has turned out that a device of the said type very successfully can be used for treatment of many medical conditions and injuries, for example athletic injuries, strains, muscular pain, joint pains, headaches, various inflammatory conditions, various skin disorders such as acne, back pains, etc., under the condition that the lights are emitted in a certain way. Hereby, the light treatment has a positive effect on the healing process for injuries, and it alleviates and/or cures various illnesses.

The device has also very successfully been used for treatment of wounds, for example of diabetic foot wounds and within geriatric care, and also within aesthetics and equestrian sports.

Hence, there is a knowledge that light treatment through the emission of a certain light in certain frequency series results in a substantially increased effect in the form of shortening of the time for the curing or alleviation of an illness.

Since the light treatment is presumed to have effect already at the cell level, it is desirable to use a dynamic light image and that the whole area on the body to be treated receives an equal amount of light over time.

In the Swedish patent no 509 717, a device for external treatment using light is disclosed, wherein the drive device for the light emitting means is arranged to control the light emitting means to emit pulsating light with a pulse length which lies within the interval of about 60% to 90% of the time between the respective start edge of two consecutive pulses.

A problem with light treatment is that all persons or animals that are treated do not respond in the same way to certain parameters, such as pulse lengths during emission of pulsating light, something that should be a consequence of the uniqueness of each individual. Thus, it has turned out that a modification of various parameters can result in improved treatment results for certain individuals. However, it is difficult to know how the parameters are to be set in order to better suit a certain individual.

The present invention solves this problem.

Hence, the present invention relates to a device for external medical treatment using light, comprising a light emitting means arranged to abut against or be held near the body of an individual as well as a drive device for the light emitting means, which light emitting means comprises light emitting diodes or the corresponding, which light emitting diodes are arranged to emit light in different wavelength intervals, wherein the drive device is arranged to control the light emitting means to emit light of one or several wavelengths during one or several predetermined times and to emit said light in a pulsating manner, which light emitting means comprises a cover and a light emitting diode plate, supporting the light emitting diodes, and wherein the light emitting diodes are positioned on said plate so that there is a certain distance between two adjacent light emitting diodes, and is characterised in that the drive device is arranged to control the light emitting means to emit pulsating light with a pulse length which lies within an interval of about 60% to 90% of the time between the respective start edge of two consecutive pulses, and arranged to vary the length of the pulses over time.

Below, an embodiment of the invention is described, partly with reference to the following drawings, where

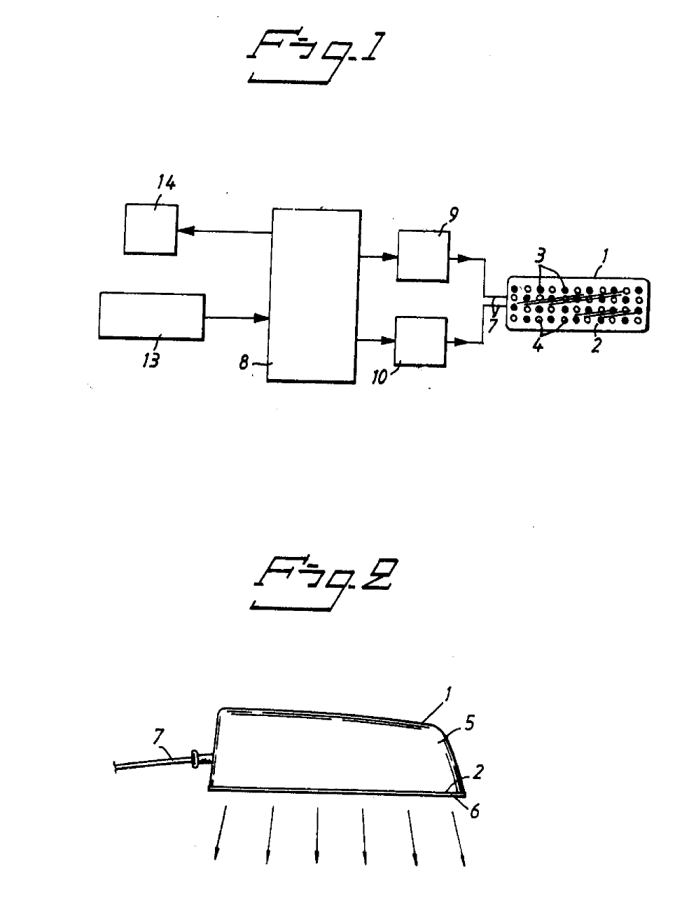

FIG. 1 schematically shows a block diagram of a device according to the invention,

FIG. 2 shows a light emitting means according to an embodiment of the invention in a side view.

FIG. 1 shows, generally, a device for external medical treatment with light, comprising a light emitting means 1 arranged to abut against or be held close to the body of an individual. The light emitting means comprises a housing 1 arranged with a transparent pane 2. Under this pane 2, inside the housing, there is a number of light emitting diodes 3, 4, see FIG. 1. The light emitting diodes 3, 4 are arranged to emit light through the pane 4 when supplied with current via a cable 7. During use, the housing 1 is held so that the pane 2 abuts against or is close to the body part in question, of for example a person to be treated. For instance, there may be a wound.

Furthermore, the device comprises a drive device 9, 10 for the light emitting means, see the block scheme in FIG. 1, wherein the light emitting means 1 is also shown from below. The drive device is arranged to control the light emitting means 1 to emit different monochromatic light of different wavelengths and in a pulsating manner, according to a predetermined pulse frequency or series of pulse frequencies.

The light emitting means 1 comprises light emitting diodes 3, 4, arranged to emit a substantially monochromatic, visible light in any of the colours violet, blue, yellow, orange, red or green, as well as infrared and other non-visible wavelengths. Which of these lights to be used depends on the illness or type of injury to be treated.

The light emitting means 1 may comprise a type of light emitting diodes 3 which are arranged to emit for instance infrared light. These are marked using filled circles in FIG. 1. Visible light may be emitted using other light emitting diodes 4, which are marked using unfilled circles in FIG. 1. The light emitting diodes for infrared light and visible light, respectively, are preferably semiconductors of the type GaAs (Gallium Arsenide). The light emitting diodes can also be arranged to emit light of other wavelengths. Thus, the light emitting means 1 may comprise light emitting diodes arranged to emit an essentially monochromatic, visible light in any of the colours violet, blue, yellow, orange, red or green, as well as infrared and other non-visible wavelengths.

The drive device for the light emitting means comprises, in a way which is known as such, a computer 8 for controlling the drive circuits 9, 10, which drive circuits are fed with signals from the computer and in turn drive the light emitting diodes via the conductor 7. To the drive device or the computer, a keyboard 13 is connected, using which the operator can key in data in order to thereby control the drive device to control the light emitting means in a desired way. Suitably, there is also a display 14 for showing the settings input via the keyboard 13.

According to the invention, the drive device 9, 10 is arranged to control the light emitting means to emit pulsating light with a pulse length lying within the interval of about 60% to 90% of the time between the respective start edge of two consecutive pulses, and arranged to vary the length of the pulses over time.

Preferably, the light pulses are square pulses.

Hereby, for each series of pulses, certain pulses will suit certain individuals while certain other pulses will suit other individuals, at the same time as all pulses will affect essentially all individuals to an effective but varying degree.

According to a preferred embodiment, the device is arranged to gradually increase the length of the pulses over time.

According to such a preferred embodiment, the device is arranged to gradually increase the pulse length form 60% of the time between the respective start edge of two consecutive pulses to 80% of the said time.

According to another preferred embodiment, the device is arranged to stepwise increase the pulse length, so that it follows a series, wherein the pulse length is 60% followed by about 70% followed by 80% of the time between the respective start edge of two consecutive pulses and arranged to thereafter repeat the series as long as the treatment continues.

According to another preferred embodiment of the invention, the device is arranged to gradually increase the pulse length over a number of steps exceeding three between 60% of the time between the respective start edge of two consecutive pulses to 80% of the said time.

It is preferred that the device is arranged to firstly emit infrared light during a predetermined first time, and thereafter visible red light during a second predetermined time.

Above, a number of preferred embodiments have been described. However, the design of the light emitting device can be varied, as well as the shape of the emitted light pulses. Therefore, the present invention shall not be considered limited to the above disclosed embodiments, but may be varied within the scope of the enclosed claims.

1. Device for external medical treatment using light, comprising a light emitting means (1) arranged to abut against or be held near the body of an individual as well as a drive device (9,10) for the light emitting means, which light emitting means comprises light emitting diodes or the corresponding (3,4), which light emitting diodes are arranged to emit light in different wavelength intervals, wherein the drive device (9,10) is arranged to control the light emitting means (1) to emit light of one or several wavelengths during one or several predetermined times and to emit said light in a pulsating manner, which light emitting means comprises a cover and a light emitting diode plate (2), supporting the light emitting diodes, and wherein the light emitting diodes are positioned on said plate so that there is a certain distance between two adjacent light emitting diodes, characterised in that the drive device (9,10) is arranged to control the light emitting means to emit pulsating light with a pulse length which lies within an interval of about 60% to 90% of the time between the respective start edge of two consecutive pulses, and arranged to vary the length of the pulses over time.

2. Device according to claim 1, characterised in that the device is arranged to gradually increase the length of the pulses over time.

3. Device according to claim 1, characterised in that the device is arranged to gradually increase the pulse length from 60% of the time between the respective start edge of two consecutive pulses to 80% of the said time.

4. Device according to claim 1, characterised in that the device is arranged to stepwise increase the pulse length, so that it follows a series where the pulse length is 60% followed by about 70% followed by 80% of the time between the respective start edge of two consecutive pulses, and is arranged to thereafter repeat the series as long as the treatment continues.

5. Device according to claim 1, characterised in that the device is arranged to gradually increase the pulse length in a number of steps exceeding three between 60% of the time between the respective start edge of two consecutive pulses to 80% of the said time.

6. Device according to claim 1, characterised in that the device is arranged to first emit infrared light during a predetermined first time, and thereafter visible light during a second predetermined time.

7. Device according to claim 2, characterised in that the device is arranged to gradually increase the pulse length from 60% of the time between the respective start edge of two consecutive pulses to 80% of the said time.

8. Device according to claim 2, characterised in that the device is arranged to stepwise increase the pulse length, so that it follows a series where the pulse length is 60% followed by about 70% followed by 80% of the time between the respective start edge of two consecutive pulses, and is arranged to thereafter repeat the series as long as the treatment continues.

9. Device according to claim 2, characterised in that the device is arranged to gradually increase the pulse length in a number of steps exceeding three between 60% of the time between the respective start edge of two consecutive pulses to 80% of the said time.

10. Device according to claim 2, characterised in that the device is arranged to first emit infrared light during a predetermined first time, and thereafter visible light during a second predetermined time.

11. Device according to claim 3, characterised in that the device is arranged to stepwise increase the pulse length, so that it follows a series where the pulse length is 60% followed by about 70% followed by 80% of the time between the respective start edge of two consecutive pulses, and is arranged to thereafter repeat the series as long as the treatment continues.

12. Device according to claim 3, characterised in that the device is arranged to gradually increase the pulse length in a number of steps exceeding three between 60% of the time between the respective start edge of two consecutive pulses to 80% of the said time.

13. Device according to claim 3, characterised in that the device is arranged to first emit infrared light during a predetermined first time, and thereafter visible light during a second predetermined time.

14. Device according to claim 4, characterised in that the device is arranged to first emit infrared light during a predetermined first time, and thereafter visible light during a second predetermined time.

15. Device according to claim 5, characterised in that the device is arranged to first emit infrared light during a predetermined first time, and thereafter visible light during a second predetermined time.
